(19) United States
(12) Patent Application Publication
(10) Pub. No.: US 2019/0070073 A1
(43) Pub. Date: Mar. 7, 2019

(54) MIXING APPARATUS AND METHOD THEREOF

(71) Applicants: Vadim ZELIKMAN, Rishon-Le-Zion (IL); Zarema ZELIKMAN, Rishon-Le-Zion (IL)

(72) Inventors: Vadim ZELIKMAN, Rishon-Le-Zion (IL); Zarema ZELIKMAN, Rishon-Le-Zion (IL)

(21) Appl. No.: 16/180,262

(22) Filed: Nov. 5, 2018

Related U.S. Application Data
(63) Continuation-in-part of application No. PCT/IL2017/050495, filed on May 4, 2017.

(30) Foreign Application Priority Data
May 4, 2016 (US) 62331467
Dec. 14, 2016 (US) 62433855

(51) Int. Cl. A61J 1/20; A61M 5/315

(52) U.S. Cl. CPC A61J 1/2093; A61J 1/2041; A61M 2005/3128; A61J 1/2027; A61M 5/31596; A61J 1/2037

(57) ABSTRACT

An apparatus for mixing a fluid material and a solid material comprises (a) a barrel; (b) a plunger displaceable within the barrel; (c) a slider disposed within the barrel between the fluid and solid materials. The slider, when reciprocatively displaced within the barrel, is permeable by the fluid material and the solved solid material under a pressure differential between the fluid and solid materials. The pressure differential is multiply exerted to the slider by the plunger such that a homogeneous mixture is obtained.

### FIELD OF THE INVENTION

The present invention pertains to a mixing apparatus and method thereof, more particularly the present invention relates to a mixing apparatus comprising a valve mechanism.

### BACKGROUND OF THE INVENTION

A variety of devices and mechanism have been proposed for mixing pharmaceutical ingredients.

EP0431779 presents a device suitable for mixing medicaments which comprises two separate containers. The first container is provided with a reservoir section and a neck section that the cross section of the neck section is less than the cross section of the reservoir. A stopper is snugly fit within the neck section adjacent to the reservoir and a spike and plunger member is snugly fit within the neck section adjacent the stopper on the side opposite the reservoir. The spike and plunger member has a midportion with a hole therein, and a plunger projecting from the midportion adapted to make contact with the stopper.

EP2361107A1 relates to a mixing syringe device for delivering a medicament or medical composition to a site of interest. The mixing syringe device includes a multi-chambered open lumen and optionally a sectioned plunger. The open lumen has at least two longitudinally oriented chambers within the interior of the lumen, and the optional sectioned plunger has at least two longitudinally oriented extensions corresponding in configuration to the at least two longitudinally oriented chambers of the open lumen and configured to be reciprocatably disposed in the chambers of the open lumen. Each compartment of the lumen is configured to store a medicament component or medical composition component in its interior. In certain embodiments, each compartment is sealed at one end by a movable stopper mechanism and sealed at the other end by a sealing mechanism capable of temporarily or otherwise opening.

EP2723426A2 presents a wet/dry auto-mixing injector having a mixing device containing at least one microfluidic channel for mixing or dissolving a dry component with a wet component stored in the injector device.

U.S. Pat. No. 7,896,836 presents a valve medicament delivery device including a housing having a chamber including coaxially aligned inlet and outlet, a medicament cartridge located within the chamber having a passage there through and membranes sealing the passage having a burst pressure of less than 10 atmospheres, a manually actuatable fluid delivery device having an outlet in fluid communication with the chamber and a manually actuated valve located between the outlet of the fluid delivery device and the chamber inlet for delivery of fluid under pressure to the valve. The medicament delivery device of this invention may be utilized to deliver a controlled unit dose of an medicament on demand by first pressurizing a pressure chamber in the pressure delivery device upstream of the valve, then opening the valve to open the membranes and express the medicament through the chamber outlet.

It therefore remains a long felt and unmet need to provide novel means and methods for mixing apparatus that provides sterile means and ease in manufacture, maintenance and handling.

### SUMMARY OF THE INVENTION

It is hence one object of the present invention to provide

It is a core purpose of the present invention to provide

Another object of the invention is to disclose

It is hence one object of the present invention to provide n apparatus for mixing a first material and a second material. The aforesaid apparatus comprises: (a) a barrel having proximal and distal portion thereof; (b) a plunger displaceable within said barrel; (c) a first slider disposed within said barrel between said first and second materials; said first slider being displaceable within said barrel along an axis thereof; (d) an orifice at said distal portion of said barrel; said orifice comprises a releasable stopper for sealing during mixing said first and second materials.

It is a core purpose of the present invention to provide the first slider, when reciprocatively displaced within said barrel, being permeable by said first material and said second material solved therewithin under a pressure differential between said first and second materials which is greater than a predetermined value; said pressure differential is multiply exerted to said first slider by said plunger such that a homogeneous mixture is obtained.

Another object of the invention is to disclose the first slider comprising a membrane having a predetermined threshold pressure to be permeable.

A further object of the invention is to disclose the pressure differential exerted by means of an action selected from the group consisting of rotating, spinning, at turning, screwing, pressing, pulling, pushing and any combination thereof.

A further object of the invention is to disclose the said first slider comprising at least one valve openable at a predetermined threshold pressure.

A further object of the invention is to disclose the action applied to said plunger coaxially in relation to said axis of said barrel.

A further object of the invention is to disclose the apparatus comprising a punching member configured for punching said first slider, such that a punched aperture providing fluid communication between said first and second materials is openable at a predetermined threshold pressure.

A further object of the invention is to disclose the plunger comprising a passage connectable to a pressure or vacuum source for creating said pressure differential between said first and second materials.

A further object of the invention is to disclose the passage which is provided with a valve.

A further object of the invention is to disclose the valve comprising two plate-like members provided with openings therewithin displaceable relative to each other; said two plate-like members have open and closed position; said open position is characterized by coincidence of said openings in one plate-like member to openings in another plate-like member; said closed position is characterized by non-coincidence of said openings in said two plate-like members therebetween.

A further object of the invention is to disclose the said first and second materials selected from the group consisting of an infusion solution, an injection solution, water, a diluent, a chemical solution, a biological solution, a medicinal solution, a dental solution, a gel, a gel-based carrier, a pharmaceutical substance or composition, herbs, food additives, food supplements, a baby formula, a dental compound and any combination thereof.

A further object of the invention is to disclose said housing comprising a retractable needle mechanism at the distal portion of said barrel.

A further object of the invention is to disclose gel-based carrier selected from the group consisting of starch, gellan gum, pregellatinized starch, modified starch, Pionil 1500 GENU GEL SVG-J, Primojel, GENU pectin, guaiacol, L-HPC(LH-31) L-HPC(LH-21), Avicel RC-591NF, alginic acid, sodium alginate, potassium alginate, ammonium alginate, calcium alginate, propane-1,2-diol alginate, agar, carrageenan, processed eucheuma seaweed, locust bean gum, guar gum, tragacanth, acacia gum, xanthan gum, karaya gum, tam gum, konjac, pectins, cellulose derivatives such as: methyl cellulose, hydroxypropyl cellulose, hydroxypropyl methyl cellulose, ethyl methyl cellulose, carboxy methyl cellulose, sodium carboxy methyl cellulose, crosslinked sodium carboxy methyl cellulose, enzymatically hydrolysed carboxy methyl cellulose, gelatin and any combination thereof.

A further object of the invention is to disclose plunger engaged to said barrel with a locking member, which enables positioning and locking said plunger in a desired location along said barrel.

A further object of the invention is to disclose plunger activated manually or in a motor-driven manner.

A further object of the invention is to disclose apparatus comprising at least one pair of unilateral valves opposite directed one-way valves openable at a predetermined. threshold pressure.

A further object of the invention is to disclose the apparatus comprising at least one valve openable at a predetermined threshold pressure.

A further object of the invention is to disclose a kit for mixing a first material and a second material; said apparatus comprising: (a) a first barrel having proximal and distal portions thereof; said first barrel having a first axis thereof; said first barrel having an orifice at said distal portion; said barrel accommodating said second material; (b) a plunger displaceable within said first barrel; (c) a second barrel having a second axis thereof; said second barrel accommodating a liquid material; (d) a second slider displaceable within said second barrel along said axis; (e) a cover connected to said second barrel.

It is a core purpose of the present invention to provide the cover, when said orifice is in fluid connection with said barrel, is permeable by said first material and said second material solved therewithin under a pressure differential between said first and second materials which is greater than a predetermined value thereat said second slider is displaced within said second barrel due to said liquid material pumped out by said plunger; said pressure differential is multiply exerted by said plunger such that a homogeneous mixture is obtained.

A further object of the invention is to disclose a method of mixing a first material and a second material. The aforesaid method comprises the steps of: (a) providing an apparatus for mixing a first material and a second material; said apparatus comprising: (i) a barrel having proximal and distal portion thereof; (ii) a plunger inserted into said proximal portion; (iii) a first slider disposed within said barrel between said first and second materials; said first slider being displaceable within said barrel along an axis thereof; (iv) an orifice at said distal portion of said barrel; said orifice comprises a releasable stopper for sealing during mixing said first and second materials; (b) creating a predetermined pressure differential between said first and second materials by reciprocatively displacing said plunger within said barrel; (c) rendering permeable said first slider by applying said predetermined pressure differential to said first slider; (d) reciprocatively flowing said first material and said second material solved therewithin under said predetermined pressure differential in a multiple manner such that a homogeneous mixture is obtained.

A further object of the invention is to disclose a method of mixing a first material and a second material. The aforesaid method comprises the steps of: (a) providing a kit for mixing a first material and a second material; said kit comprising: (i) a first barrel having proximal and distal portion thereof; said barrel having an orifice at said distal portion; said barrel accommodating said second material; (ii) a plunger inserted into said proximal portion; (iii) a second barrel having an axis thereof; said second barrel accommodating a liquid material; (iv) a second slider displaceable within said second barrel along said axis; (v) a cover connected to said second barrel; (b) adjoining said orifice to said cover; (c) said creating a predetermined pressure of said first material by reciprocatively displacing said plunger within said barrel; (d) rendering permeable said cover by applying said predetermined pressure to said cover; (e) reciprocatively flowing said first material and said second material solved therewithin under said predetermined pressure in a multiple manner such that a homogeneous mixture is obtained; said second slider is displaced within said second barrel due to said liquid material pumped out by said plunger.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings, in which

FIG. 1 is a cross-sectional perspective view of an apparatus for mixing a fluid material and a solid material;

FIGS. 2A to 2D illustrate an operation principle of an apparatus for mixing a fluid material and a solid material;

FIG. 3 is a general perspective view of an apparatus for mixing a fluid material and a solid material;

FIG. 4 is an exploded perspective view of an apparatus for mixing a fluid material and a solid material;

FIGS. 5A and 5B are perspective views of an embedded pressure valve;

FIGS. 6A to 6D illustrate an operational procedure of an apparatus for mixing a fluid material and a solid material;

FIG. 7 is a general cross-sectional view of a kit for mixing a fluid material and a solid material;

FIGS. 8A to 8D illustrate an operational procedure of a kit for mixing a fluid material and a solid material;

FIGS. 9a to 9c diagrammatically illustrate operation of a turning valve; and

FIGS. 10a and 10b diagrammatically illustrate operation of a sliding valve.

### DETAILED DESCRIPTION OF THE PREFERRED EMBODIMENTS

The present invention has been defined specifically to provide a mixing apparatus of desired substances into a predefined dosage form mixture having a required homogene.

The apparatus of the present invention is a closed system which further configured to prevent contamination, overdosing and non-sanctioned abuse of substances and/or active ingredients of a composition such as pharmaceutical composition, food supplements, baby formula, injection composition, dental compound and any known sterile composition.

The apparatus enables storing, mixing, administrating and/or delivering of one or more substances, such as pharmaceutical substances or compositions.

The present invention provides an apparatus for mixing a dosage form, comprising a housing comprising at least one lumen having a distal portion and proximal portion, the lumen comprising a plunger or piston member; and a sliding valve member; the valve configured as a separating element between a liquid phase and a solid phase and further provides a pressure difference.

In other embodiments of the present invention, the valve may be a separate element between two liquid phase substances providing a pressure difference.

The plunger member is positioned coaxially within the housing and may be engaged with the valve. The plunger rod actuates the valve.

The term ‘Dosage form’ herein refers to a solid, semi-solid, solution or a gel based. material comprising at least one drug component and/or at least one nondrug component.

The term ‘Solution’ or ‘Liquid’ herein refers primarily to a fluid material, but can also include suspensions of solids dispersed in liquids (dispersions, suspensions, colloidal mixtures), emulsions, liposomal compositions, and gasses dissolved in or otherwise present together within liquids inside the fluid-containing portions of syringes.

The liquid may be a medical solution, chemical solution, diluent, injection solution or homogeneous mixture composed of two or more substances, medication, pharmaceutical substance, water or any predefined solute.

The term ‘Solid’ refers herein to a gel based carrier material, pharmaceutical substance or composition, predefined solvent and a combination thereof.

The gel-based carrier is selected from the group consisting of starch, gellan gum, pregellatinized starch, modified starch, Pionil 1500 , GENU GEL SWG-J, Primojel, GENU pectin, guaiacol, L-HPC(LH-31) L-HPC(LH-21), Avicel RC-591NF, alginic acid, sodium alginate, potassium alginate, ammonium alginate, calcium alginate, propane-1,2-diol alginate, agar, carrageenan, processed eucheuma seaweed, locust bean gum, guar gum, tragacanth, acacia gum, xanthan gum, karaya gum, tarn gum, konjac, pectins, cellulose derivatives such as: methyl cellulose, hydroxypropyl cellulose, hydroxypropyl methyl cellulose, ethyl methyl cellulose, carboxy methyl cellulose, sodium carboxy methyl cellulose, crosslinked sodium carboxy methyl cellulose, enzymatically hydrolysed carboxy methyl cellulose, gelatine, and mixtures thereof.

The pharmaceutical substance is a solid form or liquid form of a medication, a nutritional supplement, a drag agent or herbs.

In other embodiment of the present invention, the desired solid solution or liquid material may be introduced to the apparatus thereby fill at use or may be prefilled as a part of the apparatus.

The term ‘Valve’ herein refers to a mechanical member which controls conditions such as flow, pressure, temperature, and liquid level by fully or partially opening or closing in response to mechanical activation element. Furthermore the valve regulates and facilitates a fluid passage between the proximal and distal portions of the apparatus.

The valve may have a ring-like or flange shape, The valve may be a membrane arrangement having at least one predefined aperture. The aperture is with a diameter providing a solution pathway.

The apparatus of the present invention may be a home-use apparatus which a user may activate by a single action selected from the group consisting of pressing, pulling, pushing, spinning, rotating, screwing and a combination thereof.

In other embodiment of the present invention, the liquid chamber and/or the solid chamber may be connected to a vacuum or pressure source. In these embodiments, when the solution and solid substances are held by physical forces within the apparatus (e.g., vacuum), the solution chamber and the solid chamber may translate axially within the barrel as if a unified component.

In another embodiment, the pressure within at least one chamber is in the range of about 10⁻⁶ bar to about 10⁶ bar.

In another embodiment, a method of operation of an apparatus for mixing a dosage form by (a) providing an apparatus comprising: a housing comprising at least one lumen, the housing comprising: a plunger; and At least one slidable valve member; the valve configured as a separating element between a liquid phase chamber and a solid phase chamber;

(b) pulling the plunger proximally such that a valve between the solid phase and liquid phase chambers opens;

(b) retracting, depressing or drawing back and forth the plunger to open a fluid path between the chambers, thereby actuating the valve allowing flow of liquid within the liquid phase chamber into the solid phase chamber thereby mixing the two substances;

(d) pushing the plunger to deliver mixed substances.

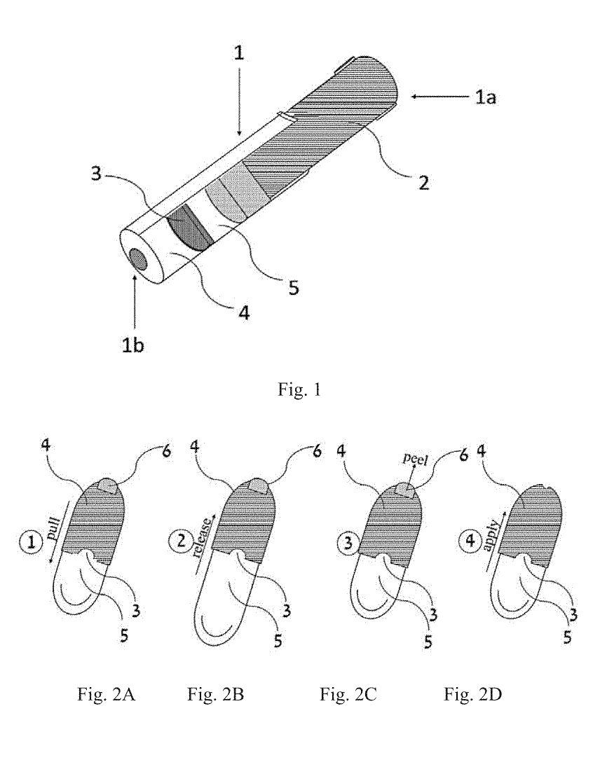

Reference is now made to FIG. 1 illustrating an apparatus for mixing a dosage form, comprising housing comprising at least one lumen having distal portion 1a and proximal portion 1b. The housing comprises plunger member 2 at the proximal portion of the housing; the plunger is movable along the lumen.

Housing 1 further comprises at least one slider 3 designed for separating liquid phase chamber 4 and solid phase chamber 5. Orifice 6 is at the distal portion 1a of housing 1. Orifice 6 may be sealed with a stopper (not shown) to maintain a pressure difference.

Distal portion 1a of housing 1 may comprise a tip having a nozzle shape for fluid extraction and for connecting a needle.

Liquid phase chamber 4 contains at least one liquid substance while solid phase chamber 5 contains at least one solid substance.

Applying a predetermined pressure differential between liquid phase chamber 4 and solid phase chamber 5 by plunger 2 renders the slider 3 permeable. In other words, the slider 3 is bifunctional because it isolates the material from the liquid material while at the predetermined pressure differential the slider is permeable for the liquid material.

In other embodiments of the present invention, slider may be snugly fit within the housing I adjacent to the lumen of the apparatus.

In other embodiments, the liquid phase chamber 4 may be the upper chamber in the proximal portion 1b and the solid phase chamber 5 may be the lower chamber in the distal portion 1a.

In other embodiments of the present invention, the plunger may be glass, metal, metal alloy, plastic or plastic rod coated with silicon oxide or barrier coatings such as parylene and the like, or any suitable material typically known in the art. The plunger configured to displaceably engage a cavity within a chamber and actuates the valve mechanism until displaced to allow sterile homogeneous mixing of substances within the chambers.

In other embodiments of the present invention, the structure of the housing is selected from the group consisting of tube shape, roller shape, rectangle shape, cylinder shape, elliptic shape, syringe shape, barrel shape, capsule shape and the like.

In other embodiments of the present invention, the plunger may be activated manually, by a handle or a pressing button which enables the movement of the plunger within the lumen.

Reference is now made to FIGS. 2A to 2D illustrating a procedure of miring a content accommodating in a dosage form 10. Slidable member 3 located between the liquid phase substance and solid phase substance accommodated in chambers 4 and 5 respectively, is actuated by the plunger member (not shown) by at least one action selected from the group consisting of at least partially rotating, at least partially spinning, at least partially turning, at least partially screwing, at least partially pressing, at least partially pulling, at least partially pushing, tapping, knocking, raping, hitting and any combination thereof.

Slidable member 3 is disposed within the lumen of the apparatus. In one embodiment, the sliding member comprises a body having outer walls which are cylindrical shaped and configured for fluid-tight contact with the inner walls of the lumen.

In other embodiments of the present invention, each of the action may be performed upon the plunger and/or the distal portion or proximal portion of the housing structure thereby allowing the content of one chamber to be transferred to the other chamber.

In other embodiments of the present invention, the valve may be actuated by pulling the plunger vertically or coaxial with the longitudinal axis of the housing,

In other embodiments of the present invention, the plunger movement forms an aperture in the valve thereby, opening a passage and allowing fluid communication between the chambers.

In other embodiments of the present invention, the plunger may be engaged to the housing of the apparatus by a locking member (not shown), which enables to position and lock the plunger in a desired location along the lumen. The locking member may be rotatable relative to the plunger from a first to a second position. The locking member may be slideable relative to the plunger when the locking member is at the first position, Furthermore the locking member prevents from the plunger sliding relative to the housing when the locking member is at the second position, thereby limiting the travel of the plunger within the lumen.

The locking member may be a fixing member preventing the movement of the locking member from the second position to the first position.

FIGS. 2A to 2D further illustrate a procedure of mixing fluid and solid content accommodating in chambers 5 and 4, respectively. In the first position (FIG. 2A). Slider member 3 is fluid communication with the liquid phase chamber. Solid phase chamber 5 is displaced from the initial position proximally to generate a substantial pressure differential for sucking the liquid material from chamber 4 into chamber 5 accommodating the solid material and solving the solid material in the liquid material.

In FIG. 2B, solid phase chamber 5 is displaced distally and the obtained solution of the solid material in the liquid material is ejected into chamber 4. Operations shown in FIGS. 2A and 2B are multiply cycled up to homogenization of the obtained solution. Then, peelable coating 6 is removed and dose form 10 is ready for application (see FIGS. 2C and 2D, respectively).

In other embodiments of the present invention, displacement of the slider member 3 from the first position (FIG. 2A) to the second position (FIG. 2B) generates fluid. pressure such as the liquid flows via slider member 3.

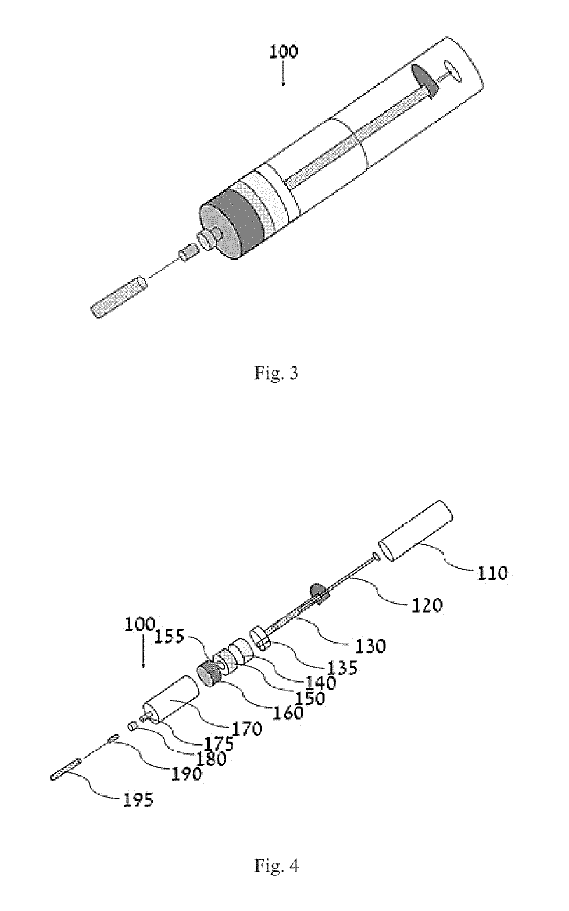

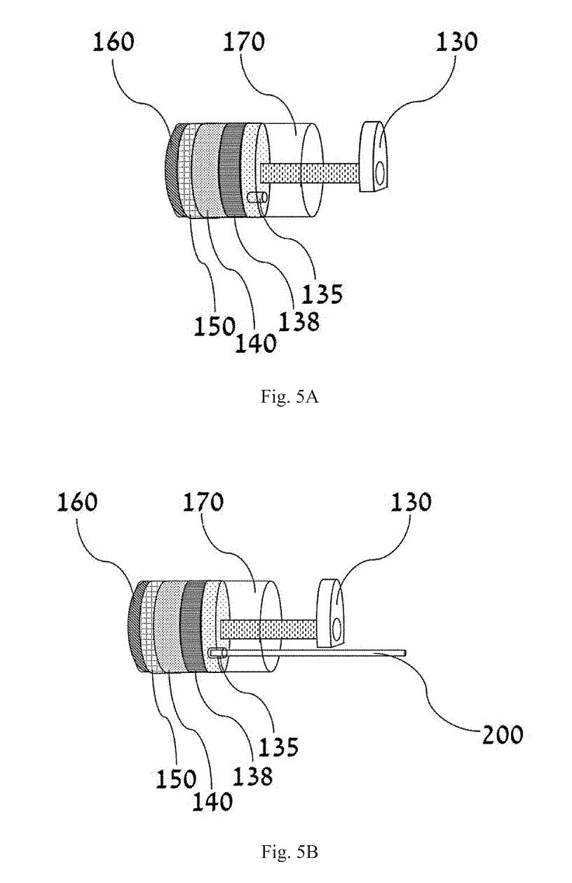

Reference is now made to FIGS. 3 and 4, presenting a general and exploded perspective views of apparatus 100 for mixing a fluid material and a solid material, Specifically, apparatus 100 comprises barrel 170 with protective cup 110. Barrel 170 is provided at distal portion with orifice 175 sealable by a stopper (cap) 175, in the process of production, the barrel 170 is provided with liquid material (gel) 160, slider 150 with membrane 155 which is optionally can be punched, solid material (powder) 140. The aforesaid elements are sealed by piston 138 which can be made of silicon. Piston 138 is movable by piston bar 130. Numeral 120 refers to a punching bar which is movable within piston bar 130. Punching bar 120 is designed for punching membrane 155 in order to facilitate flowing liquid material (gel) via slider 150 when a predetermined pressure differential is applied by displacement of piston 138. After obtaining a homogenized solution of the solid material in the liquid material, cap 180 is removed from orifice 175 and needle 190 with cover 195. After removing cover 195 from needle 190, apparatus 100 is ready for application.

Reference is now made to FIGS. 5A and 5B, illustrating pressure valve 135 embedded into piston bar 130. Pressure valve 135 is designed for controlling a pressure inside the barrel. Specifically, pressure valve 135 can be connected by means of pipe 200 to a vacuum or pressure source (not shown).

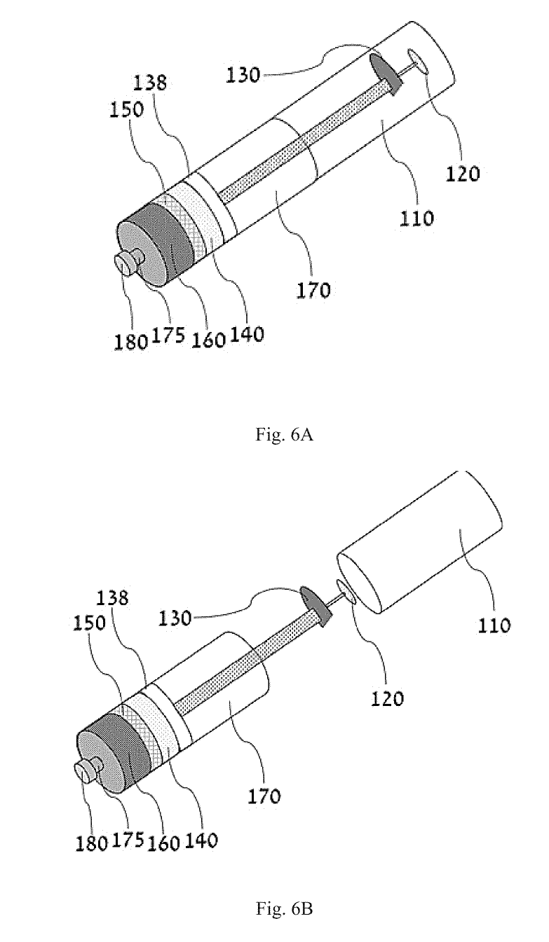

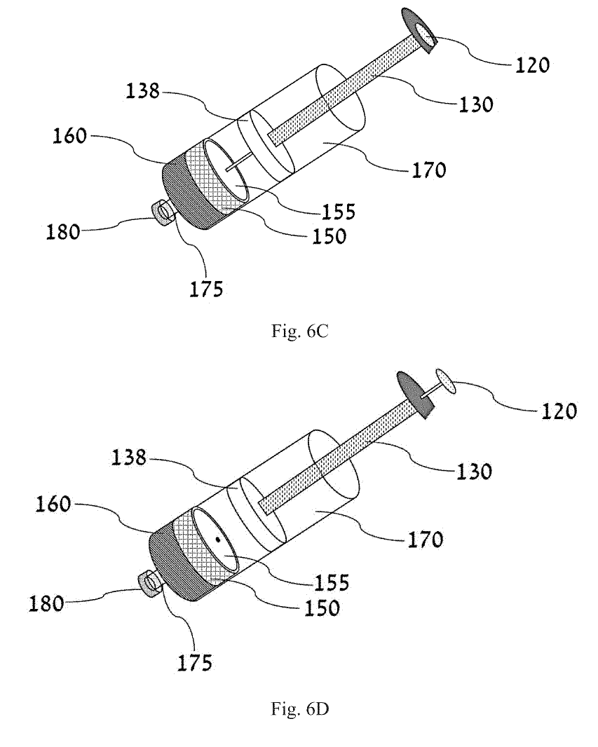

Reference is now made to FIGS. 6A to 6D showing the operation procedure of mixing liquid and solid materials in detail.

Specifically, FIG. 6A shows an initial position of the apparatus when delivered. At the first stage (FIG. 6B), protective cup 110 is removed in order to get access to piston bar 130 and punching bar 120, Then, membrane 155 is punched by punching bar 120 (FIG. 6C). Liquid material 160 is sucked via punched membrane 155 by proximally displacing piston 138 (FIG. 6D). Multiple reciprocative displacements of piston 138 result in a homogenized solution the solid material in the liquid material. Then, after removing cap 180, needle 190 is connected to orifice 175. The apparatus without needle cover 195 is ready for application.

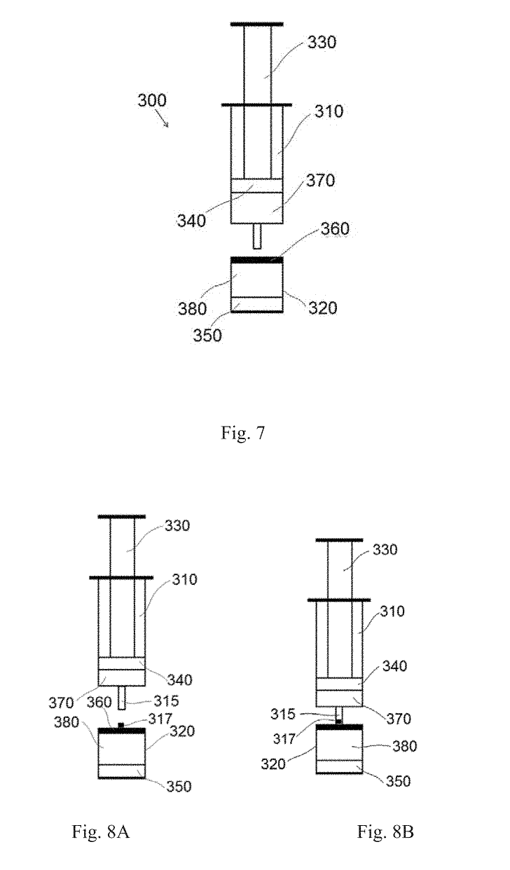

Reference is now made to FIG. 7, presenting a kit 300 for mixing a fluid material and a solid material. Kit 300 comprises first barrel 310 and second barrel 320. First barrel 310 is provided with piston bar 330 connected to piston 340. Numeral 370 refers to a solid material (powder) accommodated in barrel 310. Second barrel 320 is provided with slider 350 and closed by membrane 360 in a sealed manner. Fluid material 380 is accommodated within barrel 320.

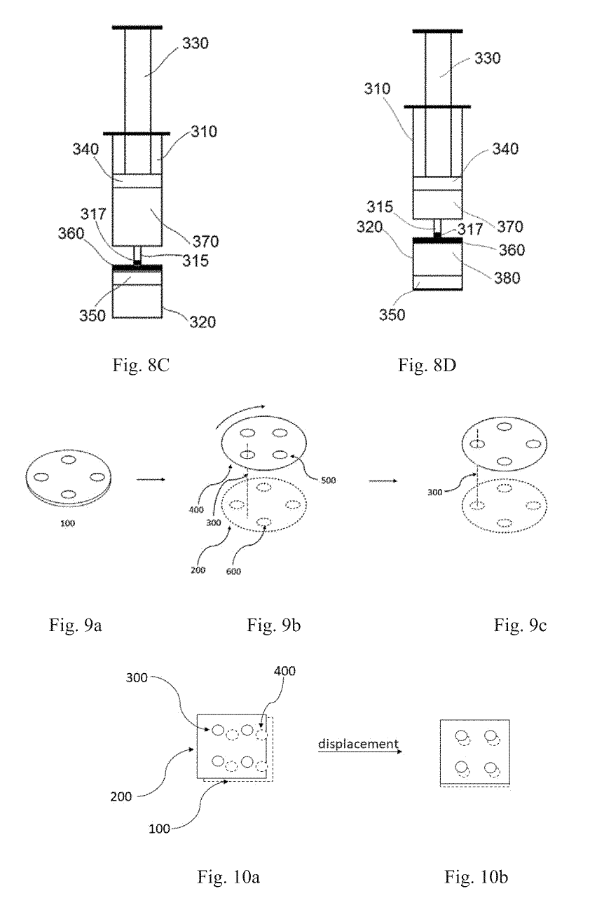

Reference is now made to FIGS. 8A to 8D, showing an operation procedure of kit 300 for mixing a fluid material and a solid material. According to one embodiment of the present invention, membrane 360 is provided with at least one valve 317.

Unidirectional and bidirectional valves are in the scope of the present invention. FIG. 8A shows kit 300 in the initial position. Then, orifice 315 is fluidly connected to valve 317 (FIG. 8B). Liquid material 380 accommodated in second barrel 320 is sucked into solid material 370 in first barrel 310. Second slider 350 is displaced within said second barrel because liquid material 380 pumped out by piston 340 (FIG. 8C).

Piston 340 is reciprocatively displaced in a multiple manner such that a homogeneous mixture or solution is obtained (FIG. 8D). After that, the obtained solution is drawn from barrel 320 into barrel 310 and ready for application.

Reference is now made to FIGS. 9a to 9c, presenting a schematic diagram illustrating a turning valve 100. In FIG. 9a, valve 100 is shown in an assembled position. FIGS. 9b and 9c present exploded views of the turning valve in closed and open positions respectively, The valve comprises two plate members 200 and 400 provided with openings 600 and 500, respectively. Numeral 300 refers to an exemplary axis of openings 500. In FIG. 9b, there is no fluid communication via the valve because axes 300 of openings 500 do not coincide with axes (not shown) of openings 600. Contrary to this, in FIG. 9c, angular displacement of two plate members 200 and 400 relative to each other provide coincidence of openings 600 and 500 and, consequently, fluid communication via the valve.

Reference is now made to FIGS. 10a and 10b presenting a sliding valve in closed and open positions, respectively, contrary to the turning valve, plates 100 and 200 are linearly displaceable relative to each other.

### EXAMPLE 1

An apparatus for mixing a dosage form is been used to prepare a homogenize mixture of purified water, strawberry flavoring, pink colorant, sucralose with Paracetamol powder formulation and pregellatinized starch fine powder.

The apparatus comprising a housing comprising at least one lumen having distal portion and proximal portion, said housing comprising:
a. a plunger member at the proximal portion of said housing; said plunger is movable along said lumen;
b. at least one slidable valve member; said valve configured as a separating element between a liquid phase chamber and a solid phase chamber; and
c. an orifice at the distal portion of said housing.

The liquid phase chamber contains purified water, strawberry flavoring, pink colorant, sucralose. The solid phase chamber contains Paracetamol powder formulation and pregellatinized starch fine powder.

The plunger is activated by depressing the plunger thereby, pushing the plunger downwardly, the plunger returns resulting from the predefined pressure formed within at least one chamber. When the plunger is pushed downward, the solution containing purified water, strawberry flavoring, pink colorant and sucralose flows directly to the slidable valve forming a mixture with Paracetamol powder formulation and pregellatinized starch fine powder within the solid phase chamber. When the plunger returns by elevated pressure within the solid phase chamber, the mixed substances flow back to liquid phase chamber via the valve.

If the apparatus is held in an upside down position, the plunger is activated by depressing the plunger thereby, pushing the plunger upwardly, the plunger returns resulting from the predefined pressure formed within at least one chamber. The pushing and/or pulling may be repeated for n (n is an integer equals to or greater than 1) times to achieve an optimal homogeneity mixture.

When the mixture is prepared the stopper located at the distal portion covering the orifice can be removed and by pressing the plunger downwards the homogeneity mixture is released and drawn out from the apparatus.

### EXAMPLE 2

The solid chamber contains Paracetamol powder formulation, gellan gum fine powder.

The liquid chamber contains purified water, strawberry flavoring, pink colorant and sucralose.

The solid chamber containing solid material may be the upper chamber or the lower chamber.

The plunger is activated by pulling the plunger upward, the plunger is depressed derived from the vacuum formed within at least one chamber. When the plunger is pulled up, the solution within the fluid phase chamber flows directly to the actuated valve allowing the content of the liquid chamber to be mixed with the content of the solid phase chamber. When the plunger returns to its first position by a vacuum formed inside the solid phase chamber, the mixed substances returns to the liquid phase chamber via the valve.

The depressing, pushing and/or pulling may be repeated for n (n is an integer equals to or greater than 1) times to achieve an optimal mixture homogeneity.

When the mixture is prepared the stopper located at the distal portion covering the orifice can be removed and by pressing the plunger downwards the homogeneous mixture is released and drawn from the apparatus.

1. An apparatus for mixing a first material and a second material; said apparatus comprising:
a. a barrel having proximal and distal portion thereof;
b. a plunger displaceable within said barrel;
c. a first slider disposed within said barrel between said first and second materials; said first slider being displaceable within said barrel along an axis thereof;
d. an orifice at said distal portion of said barrel; said orifice comprises a releasable stopper for sealing during mixing said first and second materials;
wherein said first slider, when reciprocatively displaced within said barrel, is permeable by said first material and said second material solved therewithin under a pressure differential between said first and second materials which is greater than a predetermined value; said pressure differential is multiply exerted to said first slider by said plunger such that a homogeneous mixture is obtained.

2. The apparatus according to claim 1, wherein at least one of the following is true:
a. said first slider comprises a membrane having a predetermined threshold pressure to be permeable.
b. said pressure differential is exerted by means of an action selected. from the group consisting of rotating, spinning, at turning, screwing, pressing, pulling, pushing and any combination thereof.
c. said first slider comprises at least one valve openable at a predetermined threshold pressure.

3. The apparatus according to claims 2, wherein said action is applied to said plunger coaxially in relation to said axis of said barrel.

4. The apparatus according to claim 1, wherein at least one of the following is true:
a. said apparatus comprises a punching member configured for punching said first slider, such that a punched aperture providing fluid communication between said first and second materials is openable at a predetermined threshold pressure; and
b. said plunger comprises a passage connectable to a pressure or vacuum source for creating said pressure differential between said first and second materials.

5. The apparatus according to claims 4, wherein said passage is provided with a valve.

6. The apparatus according to claims 4, wherein said valve comprises two plate-like members provided with openings therewithin displaceable relative to each other; said two plate-like members have open and closed position; said open position is characterized by coincidence of said openings in one plate-like member to openings in another plate-like member; said closed position is characterized by non-coincidence of said openings in said two plate-like members therebetween.

7. The apparatus according to claims 1, wherein at least one of the following is true:
a. said first and second materials are selected from the group consisting of an infusion solution, an injection solution, water, a diluent, a chemical solution, a biological solution, a medicinal solution, a dental solution, a gel, a gel-based carrier, a pharmaceutical substance or composition, herbs, food additives, food supplements, a baby formula, a dental compound and any combination thereof.
b. said housing comprises a retractable needle mechanism at the distal portion of said barrel.

8. The apparatus according to claim 7, wherein said gel-based carrier is selected from the group consisting of starch, gellan gum, pregellatinized starch, modified starch, Pionil 1500 , GENU GEL SWG-J, Primojel, GENU pectin, guaiacol, L-HPC(LH-31) L-HPC(LH-21), Avicel RC-591NF , alginic acid, sodium alginate, potassium alginate, ammonium alginate, calcium alginate, propane-1,2-diol alginate, agar, carrageenan, processed eucheuma seaweed, locust bean gum, guar gum, tragacanth, acacia gum, xanthan gum, karaya gum, tara gum, konjac, pectins, cellulose derivatives such as: methyl cellulose, hydroxypropyl cellulose, hydroxypropyl methyl cellulose, ethyl methyl cellulose, carboxy methyl cellulose, sodium carboxy methyl cellulose, crosslinked sodium carboxy methyl cellulose, enzymatically hydrolysed carboxy methyl cellulose, gelatin and any combination thereof.

9. The apparatus according to claims 1, wherein at least one of the following is true:
a. said plunger is engaged to said barrel with a locking member, which enables positioning and locking said plunger in a desired location along said barrel.
b. said plunger is activated manually or in a motor-driven manner.
c. said apparatus comprises at least one pair of unilateral valves opposite directed one-way valves openable at a predetermined threshold pressure.
d. said apparatus comprises at least one valve openable at a predetermined threshold pressure.
e.

10. A kit for mixing a first material and a second material; said apparatus comprising:
a. a first barrel having proximal and distal portions thereof; said first barrel having a first axis thereof; said first barrel having an orifice at said distal portion; said barrel accommodating said second material;
b. a plunger displaceable within said first barrel;
c. a second barrel having a second axis thereof; said second barrel accommodating a liquid material;
d. a second slider displaceable within said second barrel along said axis;
e. a cover connected to said second barrel;
wherein said cover, when said orifice is in fluid connection with said barrel, is permeable by said first material and said second material solved therewithin under a pressure differential between said first and second materials which is greater than a predetermined value thereat said second slider is displaced within said second barrel due to said liquid material pumped out by said plunger; said pressure differential is multiply exerted by said plunger such that a homogeneous mixture is obtained.

11. The kit according to claim 10, wherein at least one of the following is true:
a. said pressure differential is exerted by means of an action selected from the group consisting of rotating, spinning, at turning, screwing, pressing, pulling, pushing and any combination thereof.
b. said first and second materials are selected from the group consisting of an infusion solution, an injection solution, water, a diluent, a chemical solution, a biological solution, a medicinal solution, a dental solution, a gel, a gel-based carrier, a pharmaceutical substance or composition, herbs, food additives, food supplements, a baby formula, a dental compound and any combination thereof.
c. said housing comprises a retractable needle mechanism at the distal portion of said barrel.

12. The kit according to claim 11, wherein said gel-based carrier is selected from the group consisting of starch, gellan gum, pregellatinized starch, modified starch, Pionil 1500, GENU GEL SVSG-J, Primojel, GENU pectin, guaiacol, L-HPC(LH-31) L-HPC(LH-21), Avicel RC-59 INF, alginic acid, sodium alginate, potassium alginate, ammonium alginate, calcium alginate, propane-1,2-diol alginate, agar, carrageenan, processed eucheuma seaweed, locust bean gum, guar gum, tragacanth, acacia gum, xanthan gum, karaya gum, tara gum, konjac, pectins, cellulose derivatives such as: methyl cellulose, hydroxypropyl cellulose, hydroxypropyl methyl cellulose, ethyl methyl cellulose, carboxy methyl cellulose, sodium carboxy methyl cellulose, crosslinked sodium carboxy methyl cellulose, enzymatically hydrolysed carboxy methyl cellulose, gelatin and any combination thereof.

13. The kit according to claims 10, wherein said plunger is engaged to said barrel with a locking member, which enables positioning and locking said plunger in a desired location along said barrel.

14. The kit according to claims 10, wherein said plunger is activated manually or in a motor-driven manner.

15. The kit according to claims 10, wherein said cover comprises a membrane having a predetermined threshold pressure to be permeable.

16. The kit according to claims 10, wherein said cover comprises at least one pair of unilateral valves opposite directed one-way valves openable at a predetermined threshold pressure.

17. The kit according to claims 10, wherein said cover comprises at least one valve openable at a predetermined threshold pressure.

18. The apparatus according to claims 10 comprising a valve comprises two plate-like members provided with openings therewithin displaceable relative to each other; said two plate-like members have open and closed position; said open position is characterized by coincidence of said openings in one plate-like member to openings in another plate-like member; said closed position is characterized by non-coincidence of said openings in said two plate-like members therebetween.

19. A method of mixing a first material and a second material; said method comprising the steps of:
a. providing an apparatus for mixing a first material and a second material; said apparatus comprising;
i. a barrel having proximal and distal portion thereof;
ii. a plunger inserted into said proximal portion;
iii. a first slider disposed within said barrel between said first and second materials; said first slider being displaceable within said barrel along an axis thereof;
iv. an orifice at said distal portion of said barrel; said orifice comprises a releasable stopper for sealing during mixing said first and second materials;
b. creating a predetermined pressure differential between said first and second materials by reciprocatively displacing said plunger within said barrel;
c. rendering permeable said first slider by applying said predetermined pressure differential to said first slider;
d. reciprocatively flowing said first material and said second material solved therewithin under said predetermined pressure differential in a multiple manner such that a homogeneous mixture is obtained.

20. A method of mixing a first material and a second material; said method comprising the steps of:
a. providing a kit for mixing a first material and a second material; said kit comprising:
i. a first barrel having proximal and distal portion thereof; said barrel having an orifice at said distal portion; said barrel accommodating said second material;
ii. a plunger inserted into said proximal portion;
iii. a second barrel having an axis thereof; said second barrel accommodating a liquid material;
iv. a second slider displaceable within said second barrel along said axis;
v. a cover connected to said second barrel;
b. adjoining said orifice to said cover;
c. said creating a predetermined pressure of said first material by reciprocatively displacing said plunger within said barrel;
d. rendering permeable said cover by applying said predetermined pressure to said cover;
e. reciprocatively flowing said first material and said second material solved therewithin under said predetermined pressure in a multiple manner such that a homogeneous mixture is obtained; said second slider is displaced within said second barrel due to said liquid material pumped out by said plunger.
